(19) United States
(12) Patent Application Publication
(10) Pub. No.: US 2026/0364111 A1
(43) Pub. Date: Nov. 19, 2026

(54) COMPOSITIONS BASED ON A MIXTURE OF BIOACTIVE MOLECULES AND EXOSOMES FOR USE IN THE TREATMENT OF CONDITIONS REQUIRING TISSUE REPAIR AND REGENERATION

(71) Applicant: BIOMEDICAL RESEARCH S.R.L., Catania (IT)

(72) Inventor: Filippo SALVAGGIO, Catania (IT)

(21) Appl. No.: 19/132,340

(22) PCT Filed: Nov. 27, 2023
(86) PCT No.: PCT/IB2023/061927

§ 371 Date: May 22, 2025

(30) Foreign Application Priority Data
Nov. 25, 2022 (IT) 102022000024306

(51) Int. Cl. A61K 35/20; A61K 9/00; A61K 31/198; A61K 31/728; A61K 33/42; A61K 35/19; A61P 17/02

(52) U.S. Cl. CPC A61K 35/20; A61K 9/0014; A61K 9/0019; A61K 31/198; A61K 31/728; A61K 33/42; A61K 35/19; A61P 17/02

(57) ABSTRACT

Topical or injection compositions for use in the treatment of conditions requiring tissue repair and regeneration in humans and animals include a mixture of bioactive molecules and exosomes. The mixture of bioactive molecules is isolated from colostrum.

The present invention relates to topical or injection compositions based on a mixture of bioactive molecules (e.g., isolated from biological tissues and fluids of mammals: placenta, colostrum, and prepartum serum) and exosomes for use in the treatment of conditions requiring tissue repair and regeneration, in humans and animals.

It is known that a mixture of bioactive molecules mainly containing growth factors, cytokines, stem cell stimulating factors, complement proteins, antibacterial and antiviral factors, immunoglobulins can be isolated from some tissues and biological fluids of mammals such as the placenta and prepartum serum, but in particular from colostrum.

### Growth Factors and Cytokines

Growth factors and cytokines are chemical messengers which mediate intracellular communication to regulate a variety of cellular functions. They can also exert their action through endocrine mechanisms, but, due to the short half-life and slow diffusion into intercellular spaces thereof, they typically act locally as autocrine or paracrine signals.

These messengers bind to receptors present on the cell surface thus initiating, at the membrane level and in the intracellular environment, a cascade of events involved in the signal transduction process.

Often, the terms “cytokines” and “growth factors” are used to indicate these molecules in a superimposable and indistinct manner, however, cytokines behave like growth factors for many cell types, for others as regulators of cell division and apoptosis or cell death and also play an important role in the innate and specific immune response and in the inflammatory phase of the tissue repair process.

Growth factors and cytokines are thus capable of stimulating a variety of cellular processes s including proliferation, migration, differentiation, and morphogenesis during the development or healing of injured tissues.

Three main groups of growth factors are distinguished by structural homologies: insulin-like growth factors, fibroblast growth factors and transforming growth factors.

The growth factors belonging to the first group, also referred to as somatomedins, are represented by IGF1 and IGF2 (Insulin-like Growth Factor 1 and 2), mainly synthesized by the liver and with an insulin-like structure. IGF1 and IGF2 carry out an insulin-like action, regulating carbohydrate homeostasis, and exert an anabolic action on bone metabolism similar to that of growth hormone (GH). Moreover, they regulate cell proliferation and differentiation, especially at the cartilage and muscle levels.

Endothelial cells produce and respond to Fibroblast Growth Factors (FGFs): these are involved in the wound healing process by stimulating angiogenesis, collagen synthesis, epithelial cell migration and extracellular matrix synthesis. Keratinocyte Growth Factor, known as KGF, also belongs to the same family of growth factors, playing an important role in the re-epithelialization process, stimulating the proliferation and differentiation of keratinocytes.

Transforming Growth Factors TGF-α and TGF-β are instead multifunctional cytokines that play a fundamental role in tissue regeneration.

TGF-β produced by most granulation tissue cells, including activated macrophages, stimulates fibroblast migration and proliferation, increases collagen and fibronectin synthesis, and counteracts extracellular matrix (ECM) degradation by inhibiting metalloproteinases. It acts in an autocrine manner, regulating the production thereof and stimulating monocytes to produce other growth factors, such as fibroblast growth factor (FGF), Platelet-Derived Growth Factor (PDGF) and Tumor Necrosis Factor α (TNF-α). TGF-β is also an anti-inflammatory cytokine which is used to limit and resolve inflammatory responses, inhibiting lymphocyte proliferation and the activity of other leukocytes.

TGF-α is a peptide which is structurally correlated to Epidermal Growth Factor (EGF) and with a similar action. EGF acts on epithelial cells, stimulating cell mitosis and therefore the re-epithelialization, but also on fibroblasts, smooth muscle cells and endothelial cells, promoting angiogenesis and the process of forming new blood vessels starting from pre-existing vessels, a fundamental process in wound healing. The newly formed vessels must be stabilized by the intake of smooth muscle cells and by the deposition of connective tissue. PDGF, produced by platelets, and TGF-β participate in this stabilization process: the former recalls smooth muscle cells and acts by activating the expression of TGF-β, which suppresses endothelial proliferation and migration and increases ECM protein production.

Vascular Endothelial Growth Factors (VEGF) also make an important contribution in the angiogenesis process, stimulating the migration and proliferation of endothelial cells and contributing to the formation of the vascular lumen. TNF-α, mentioned above, belongs to the category of inflammatory cytokines mainly produced by cells of the monocyte-macrophage system. It plays a role in the wound repair process as a stimulator of angiogenesis.

Both the cytokines linked to innate immunity, such as TNF-α, IL-1, MCP-1 and IL-15, and those linked to the regulation of acquired immunity, such as IL-2, IL-4, IFN-γ, IL-9 and IL-17, have been highlighted as important components of the mixture of bioactive molecules.

The cytokine IL-17, present in high concentrations in the mixture and produced in mammals by a specific class of cells referred to as Th17, allows the release of chemokines and growth factors from mesenchymal cells; moreover, the modulatory action thereof has also been demonstrated in many diseases such as asthma, Crohn's disease, multiple sclerosis, psoriasis and rheumatoid arthritis. The cytokine IL-17 can thus be considered one of the most important mediators of inflammatory, autoimmune, fungal and viral diseases. High levels of IL-15, which induces the proliferation of T, B and natural killer (NK) cells, stimulating the maturation of the immune system and also exerting an important anticancer activity, were also measured in the mixture.

### Stem Cell Stimulating Factors

Stem Cell Factor (SCF) is involved in the development of hematopoietic, gonadal, and pigmented cell lines. It has a very wide range of activities with direct effects on the development of myeloid and lymphoid cells and potent synergistic effects with other growth factors such as GM-CSF, IL-7 and erythropoietin.

Granulocyte Colony Stimulating Factor (G-CSF) and Granulocyte Macrophage Colony Stimulating Factor (GM-CSF) are key substances for the proliferation, maturation and expansion of bone marrow progenitor cells. These factors are capable of binding specific receptor proteins located on the surfaces of hematopoietic stem cells, present in bone marrow. The bond between receptor and colony-stimulating factor activates intracellular metabolic pathways that cause the cells to proliferate and differentiate into a specific type of blood cell.

The role thereof as stimulating factors in stem cells is thus now well defined.

### C3a/C4a Complement Proteins

Complement proteins are components of the homonymous system which supports the body in immune and inflammatory responses.

They are mostly synthesized (90%) by the liver, with the rest instead synthesized by fibroblasts, monocytes and macrophages.

The system consists of over 60 proteins, but only about thirty of these circulate in the blood. In fact, these molecules are present as inactive precursors in interstitial fluids and serum and are activated when a foreign agent triggers the body's endogenous response, which thus produces antibodies to neutralize the threat.

There are nine main proteins of the complement system, indicated from C1 to C9. These, together with all the other proteins in the system, interact in a cascade pattern, forming complexes which work to support the immune system in different situations such as infections, inflammation or apoptosis.

Fractions C3/a and C4/a are the most represented in the mixture of bioactive molecules isolated from mammalian tissues and biological fluids.

### Antibacterial and Antiviral Factors

Lactoferrin and Transferrin are among the main proteins involved in iron metabolism. Lactoferrin, in particular, has several other functions, including acting as a direct antimicrobial agent against a wide range of bacteria and viruses. Moreover, Lactoferrin has been shown to stimulate the growth of several cell lines in vitro, including fibroblasts and intestinal epithelial cells, suggesting that the presence thereof, for example in colostrum, can be important in regulating intestinal development in infants.

Lysozyme is an enzyme of the glycosidase group provided with a bacteriolytic action and contained in nasal mucus, saliva, tears, tissues, blood serum and breast milk. It hydrolyzes the glycosidic bond between N-acetylmuramic acid and N-acetyl-glucosamine, major constituents of the cell wall mucopeptides of many gram-positive bacteria, causing the lysis thereof. It is synthesized by white blood cells and macrophages and, with the proteolytic activity thereof, participates in the defense of the body.

Lactoperoxidase is a peroxidase enzyme secreted by the mammary, salivary, and other mucosal glands, including the lungs, bronchi, and nose, which acts as a natural first line of defense against bacteria and viruses.

Lactoperoxidase catalyzes the oxidation of various inorganic and organic substrates by hydrogen peroxide. The oxidized products, derived from the action of this enzyme, have potent and non-specific bactericidal and antiviral activities, including the destruction of the influenza virus.

### Immunoglobulins

Immunoglobulins are glycoprotein molecules with antibody activity, produced by B lymphocytes in response to antigenic stimulation: the final cell figure in the series of transformations which the B lymphocyte undergoes is the plasma cell capable of secreting mature antibodies, which represent the effector molecules of humoral immunity.

5 classes of immunoglobulins are recognized: IgG, IgA, IgM, IgD and IgE, distinguishable based on the structure of the molecule and identifiable in clinical practice based on the different molecular weight by means of electrophoresis.

Immunoglobulins belonging to the same class have identical physicochemical features (allowing, for example, the passage of the antibody molecule through the placenta or breast milk) and the same effector functions, such as the ability to bind to immune cells (opsonization) or to activate the complement.

IgGs, which form 85% of Immunoglobulins present in the blood, activate complement and promote opsonization. IgMs, which form up 5-10% of Immunoglobulins present in the blood, are the first to form in response to an unknown antigen. IgAs, which form 10-20% of the Immunoglobulins present in the blood, represent the highest portion of Immunoglobulins present in exocrine secretions (milk, saliva), forming the body's first defense barrier against the entry of pathogenic germs. IgDs form less than 1% of Immunoglobulins and the biological function thereof is not fully known. IgEs, physiologically present in minimal amounts in the blood, are responsible for allergic reactions, bind to basophilic leukocytes and mast cells and cause the degranulation thereof.

### Other Components

The mixture of bioactive molecules also contains APLN peptides (not properly recognized as growth factors but with functions similar to proteins belonging to this class), non-peptide trophic factors and vitamins, in particular vitamin A, vitamin E, vitamin B12, but also traces of other vitamins, such as D and provitamin A (beta carotene).

Tryptic inhibitors are also present exclusively in the colostrum-derived mixture. These compounds allow bioactive molecules, when administered orally, not to be broken down by gastric digestive enzymes and to reach the intestine intact, where they exert the activity thereof or are absorbed. Some recent researches have further shown how these inhibitors can prevent the adhesion of the bacterium responsible for gastric ulcer (Helicobacter pylori) to the stomach walls.

The main bioactive molecules described above are shown in FIG. 1 (growth factors and stem cell stimulating factors), FIG. 2 (cytokines) and FIG. 3 (immunoglobulins, antibacterial and antiviral factors, complement proteins).

### SUMMARY OF THE INVENTION

The inventors of the present patent application have surprisingly found that by combining a mixture of colostrum-isolated bioactive molecules with exosomes, its activity is unexpectedly enhanced in all fields of application.

### OBJECT OF THE INVENTION

In a first object, the present invention relates to a composition comprising a mixture of colostrum-isolated bioactive molecules and exosomes.

According to a particular aspect, the exosomes are obtained from colostrum.

In a second object of the invention, formulations comprising the composition of the invention are described.

In a third object, it is described the medical use of the disclosed composition.

The multiple medical uses of the disclosed composition represent further aspects of the present invention.

### BRIEF DESCRIPTION OF THE DRAWINGS

FIGS. 1, 2 and 3 list the main factors present in the mixture of bioactive molecules isolated from mammalian tissues and biological fluids and in particular from colostrum (growth factors and stem cell stimulating factors-cytokines-immunoglobulins, antibacterial and antiviral factors, complement proteins).

FIG. 4 shows the flow diagram for the exosome purification procedure.

FIG. 5 shows the protocol for the production of PRP.

FIG. 6 shows the protocol for the production of PRF.

FIG. 7 shows the protocol for the production of PRGF.

FIGS. 8 to 20 show the results obtained with the MTT Assay using different cell lines.

FIG. 21 shows a depiction of the Wound Healing Assay.

FIG. 22 shows the results obtained with the Wound Healing Assay at 24, 48 and 72 hours.

FIG. 23 shows the results obtained with the “Test on a biological model of human hair follicle”.

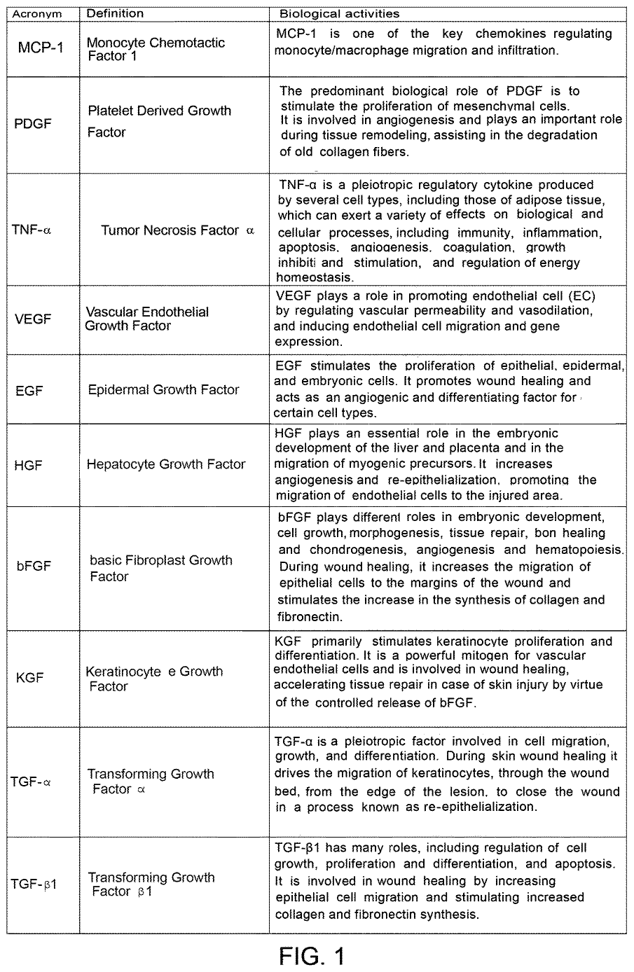

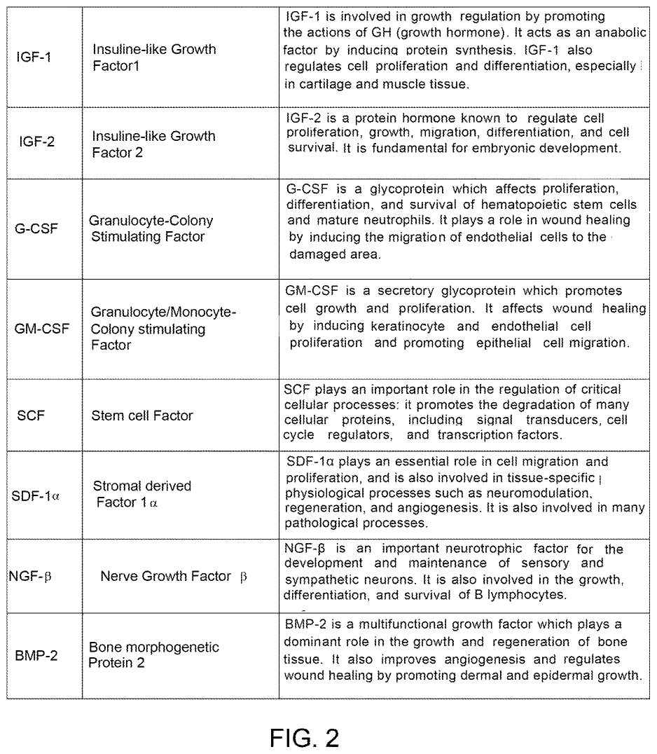

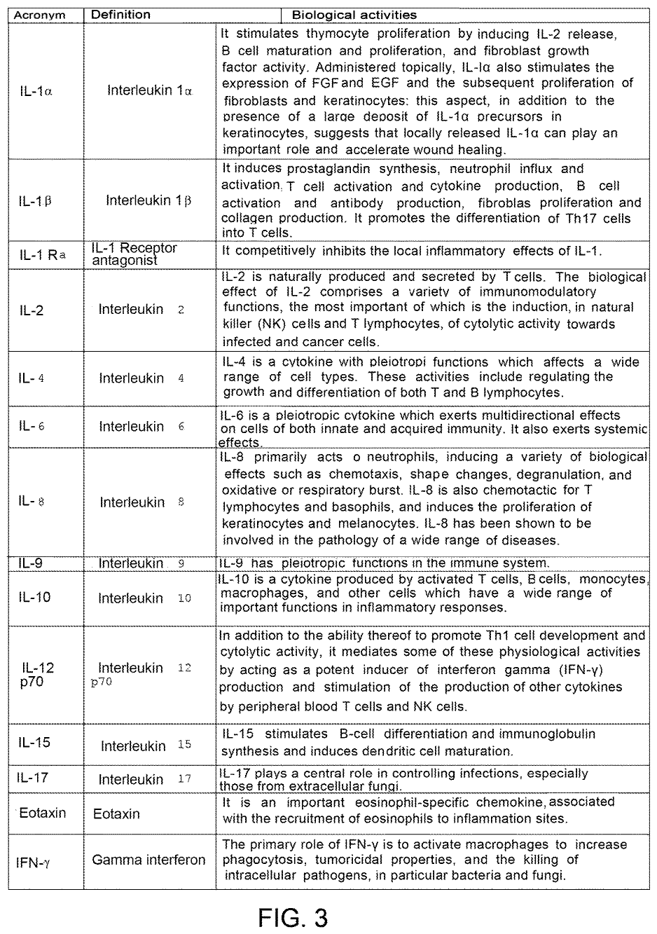

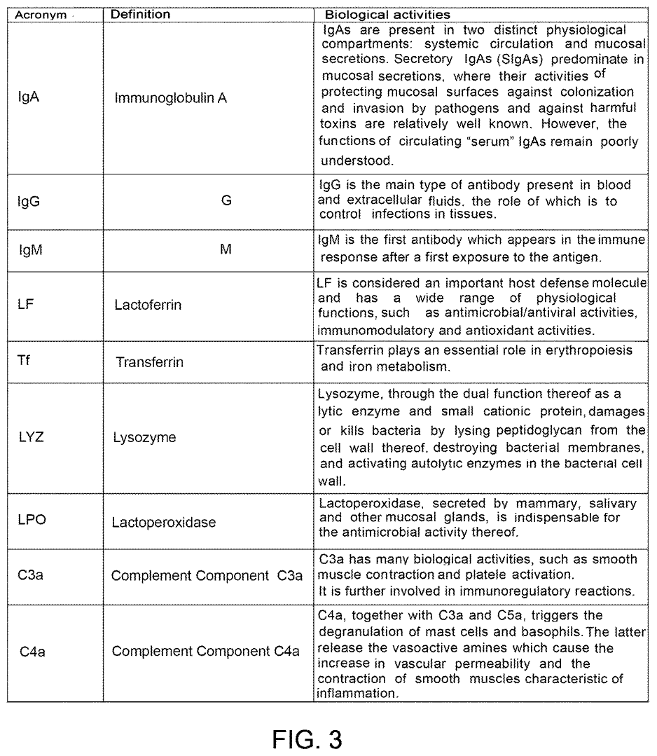

### DETAILED DESCRIPTION OF THE INVENTION

According to a first object of the invention, the composition described comprises a mixture of bioactive molecules and exosomes.

For the purposes of the present invention, the mixture of bioactive molecules is isolated from colostrum.

Alternative sources to colostrum are represented by other tissues and biological fluids of mammals.

In particular, it is the placenta and the prepartum serum.

Such a mixture of bioactive molecules comprises: growth factors, cytokines, stem cell stimulating factors, C3a/C4a complement proteins, antibacterial and antiviral factors, immunoglobulins and possibly also APLN peptides (not properly recognized as growth factors but with functions similar to proteins belonging to this class), non-peptide trophic factors and vitamins, in particular vitamin A, vitamin E, vitamin B12, but also traces of other vitamins, such as D and provitamin A (beta carotene).

Tryptic inhibitors are also present in the colostrum-derived mixture.

### Mixture of Colostrum-Isolated Bioactive Molecules

It is possible to obtain a mixture of biological factors from the colostrum with an appropriate method, comprising: growth factors, cytokines, stem cell stimulating factors, complement proteins C3a/C4a, antibacterial and antiviral factors, immunoglobulins.

The colostrum used for the purposes of the present invention is obtained from mammals, including humans, and preferably is obtained from cattle. In fact, colostrum from herbivores has been found to contain several bioactive molecules to a greater extent.

Bovine colostrum is the richest source of biological factors and therefore the most advantageous. In particular, dairy breeds have been shown to produce colostrum with the highest concentration of growth factors, cytokines, stem cell stimulating factors, complement proteins, antibacterial and antiviral factors, immunoglobulins. Taking the immunoglobulin concentration as an indicator of colostrum quality, the breed which produces the colostrum richest in bioactive factors is Jersey with 9.0% immunoglobulins, followed by Ayrshire with 8.1%, Brown Swiss with 6.6%, Guernsey with 6.3%, and Friesian (Holstein) with 5.6%.

These biological factors are highly conserved in phylogenesis and therefore have a considerable cross-reaction activity between the various species, therefore it is possible to use factors isolated from other mammals such as cattle, horses, camelids, etc. on humans.

The colostrum used for the purposes of the present invention is preferably collected before the newborn has had the opportunity to nurse and empty the udder of the first fraction produced, regardless of the time elapsed from the moment of birth. In fact, the greatest concentration of active ingredients is present precisely in the first fraction of colostrum produced by the udder. In a preferred aspect, the cows are preferably at the second or third birth.

Advantageously, the method for preparing the colostrum mixture rich in biological factors does not include steps or operations which could damage such factors, reducing the activity thereof.

For example, no heat sterilization steps are carried out which can lead to the loss of large amounts of biological factors in the final product following the degradation caused by high temperatures. Or, the separation of casein, necessary as it can cause allergic reactions, cannot include a precipitation step at low pH values which can lead to the denaturation of many proteins, including various biological factors present in the colostrum.

Therefore, for the preparation of the mixture of colostrum-isolated bioactive molecules according to the present invention, extraction processes which allow obtaining a mixture of factors in a biologically active form will be used.

Processes which can be employed for the preparation of the mixture rich in biological factors are described for example by P. Sacerdote et al. (“Biological components in a standardized derivative of bovine colostrum”—Journal of Dairy Science, Volume 96, Issue 3, 1 Mar. 2013, pages 1745-1754) and in international patent application WO2017134559. Other extraction processes may be used, as long as they meet the above requirements.

In a preferred aspect of the invention, the colostrum isolated mixture rich in biological factors is obtained according to the process described by P. Sacerdote et al., comprising the steps of:
1) dilution;
2) skimming;
3) ultrafiltration;
4) dialysis;
5) microfiltration.

In another preferred aspect of the invention, in step 1), a 100 kg amount of colostrum is placed in a reactor and diluted 1:10 with deionized water added with 0.9% NaCl.

In a further preferred aspect of the invention, in step 2) of skimming, the whole mass is centrifuged at 12,400 g at a temperature of 20-25 °C to remove the fat part.

According to a particularly preferred aspect of the invention, in step 3), the previously obtained liquid phase is subjected to ultrafiltration through a membrane with 300 KDa cut-off, always maintaining the temperature of 20-25 °C, to remove the large proteins and pathogenic microorganisms. For example, the caseins are removed from the large proteins.

In another particularly preferred aspect of the invention, in step 4) of dialysis, the colostrum isolate mixture rich in biological factors thus obtained is then dialyzed with a 5 KDa membrane to remove any preservatives or drug residues present in the starting colostrum and especially the lactose in solution.

In a further particularly preferred aspect of the invention, in the microfiltration step 5), the product obtained is subjected to a series of sterilizing cross-flow filtration steps with 0.2 μm membranes and then frozen.

Therefore, the product obtained is characterized by a mixture of biological factors, also comprising the immunoglobulins (IgG, IgA and IgM) provided with a natural specificity against many bacteria and viruses.

As the last step of the preparatory process, the mixture is subjected to a lyophilization step.

A sterile, preservative-free, hypoallergenic powder is thus obtained with very high solubility and with the highest possible concentration of active factors.

In a preferred aspect, to evaluate the quality of the processes implemented to obtain a standardized final product, some key colostrum growth factors are used as markers, which are quantified by exploiting individual known ELISA tests.

If it is desired to obtain a mixture of active factors isolated from colostrum which can be used in injectable form in those countries where health legislation prohibits the injection administration of heterologous immunoglobulins, it is possible to add further steps to the method described above which allow obtaining a product deprived of the immunoglobulins. The product thus obtained has a high proliferative and regenerative activity because, with the same weight, the content of bioactive molecules is much higher.

According to another preferred aspect of the present invention, the process for preparing the mixture of colostrum-isolated active factors thus comprises a further purification step. In particular, such a step is carried out on the concentrated product before freezing and lyophilization, and is characterized by an IgG and IgA depletion step, which, in a preferred aspect of the invention, is carried out by means of affinity chromatography.

The colostrum purification step further comprises a step of IgM depletion, for example carried out by tangential filtration or cartridge filters.

METHOD: in a preferred aspect in the method of the present invention, affinity chromatography is used by means of:
1. CaptureSelect™ IgG-Fc (ms) Affinity Matrix (Thermo Fisher Scientific) or equivalent product. This system has been specifically designed to purify IgGs from different species (human, mouse, rat, rabbit, cow, horse, and sheep) by high affinity binding to the Fc region of IgG.
2. CaptureSelect™ Bovine IgA Affinity Matrix (Thermo Fisher Scientific) or equivalent product. This system has been specifically designed for the purification of bovine IgA from whey/colostrum. In CaptureSelect™ Bovine IgA Affinity Matrix, affinity ligands are created using proprietary technology based on single domain antibody fragments derived from camelids.

In a further preferred aspect of the present invention, the depletion of IgMs and immunoglobulin aggregates occurs by means of a tangential flow filtration step which uses membranes with a molecular cut-off of about 750 kDa-0.02 μm, or by using cartridge filters with pores of the same size. More in detail, the pentamers of the M immunoglobulins, which have an average molecular weight of 800-900 kDa, can be removed by tangential filtration, or cartridge filters, using membranes of appropriate size, while the other bioactive factors, with a lower molecular weight, can cross them without being retained. Different filtration methods were tested as well as a variety of filters and the best results in terms of IgM and aggregate depletion were obtained using cartridge filters with pores of 750 kDa-0.02 μm, type ENTEGRIS Savana® PS cartridge filter 0.02 micron or equivalent product.

In a particularly preferred aspect, again in relation to the injective use of the product, a further purification step of the colostrum isolate mixture is necessary to deprive it of the possible presence of bacterial endotoxins (exogenous pyrogens).

In another particularly preferred aspect of the present invention, the removal of the endotoxins occurs by means of specific kits which use an affinity chromatography, such as the Bio-Rad® Proteus Endotoxin Removal kits or equivalent products. Alternatively, cartridge filters with directly incorporated positively charged nylon membrane can be used which, by strongly interacting with the negatively charged endotoxins, can adsorb consistent concentrations of the latter present in solution. The best results in terms of endotoxin removal were obtained using BEA Technologies POSINYL nylon 6.6 membrane cartridge filters or equivalent product.

In particular, all these further steps, necessary to purify the mixture for injective use, are carried out on the concentrated product before freezing and lyophilization.

The process was developed with a view to industrial application. All the process steps are transferable with a view to a large-scale purification process.

The mixture of bioactive molecules according to the present invention is much more effective in terms of proliferative and regenerative properties, with respect to the constituent factors tested alone or in small artificially-created groups. In fact, the growth factors and other bioactive molecules naturally present in the mixture do not act individually, but fully carry out the action thereof only if they are in an appropriate combination, determined by nature during evolution, where all the components work in unison like the different instruments of an orchestra.

In fact, the interaction between growth factors, cytokines, stem cell stimulating factors, complement proteins, antibacterial and antiviral factors, and exclusively for oral administration, immunoglobulins, determines the initiation of a cascade of intracellular biochemical signals, followed by activation or inhibition of genes which control different cellular functions, and in particular cell proliferation and differentiation.

The close correlation between these bioactive molecules in inducing and modulating an effective regenerative response by injured tissues emerges from the above. When lacking entirely or in part, the action of some of these factors will have a null, partial or even harmful response.

Moreover, all the bioactive molecules described are genetically highly conserved in the different species and thus for humans it is possible to use, for reparative and regenerative purposes, the biological factors isolated from other species of mammals such as cattle, horses, camels, etc.

### Exosomes

For the purposes of the present invention, the disclosed composition further comprises exosomes.

Exosomes represent the most extensively studied group among the three major subgroups of extracellular vesicles or EVs (exosomes, microvesicles, and apoptotic vesicles or ApoEVs). Among these, exosomes are the smallest vesicles, with a variable diameter of 50-150 nm and a cup-shaped morphology when viewed under an electron microscope.

The biogenesis process of the exosome includes several steps: 1) the inward folding of a section of the plasma membrane forms the early endosomes; 2) the first endosomes mature into late endosomes and then into multivesicular bodies (MVBs) characterized by intraluminal vesicles (ILVs); 3) once they reach maturity, these MVBs are pulled by means of a network of microtubules and cytoskeletons towards the cell membrane where, by exocytosis, they will fuse therewith, releasing the ILVs into the extracellular environment which will become exosomes. This process of biogenesis and exosome formation is finely directed by the Endosomal Sorting Complexes Required for Transport (ESCRT) and accessory proteins.

Exosomes possess a specific molecular load which includes membrane proteins, cytosolic and nuclear proteins, extracellular matrix proteins, lipids, nucleic acids (mRNA, miRNA and DNA), growth factors, cytokines and enzymes, underlining the involvement thereof in the regulation, by means of different molecular mechanisms, of various biological functions.

The absorption of exosomes depends on the interactions between the proteins expressed on the surface thereof and the recipient cells. Several studies suggest that adhesion molecules on the surface of exosomes, such as tetraspanins, glycoproteins, and integrins, determine which cells can “accept” exosomes.

Exosomes are secreted by most cell types including immune cells (B cells, T cells), neuronal cells, epithelial cells, endothelial cells, embryonic cells, and mesenchymal stem cells (MSCs).

Exosomes have been detected in almost all body fluids, under both physiological and pathological conditions, including: urine, blood, serum, breast milk, amniotic fluid, cerebrospinal fluid, saliva, bile, and lymph.

Exosomes are also contained in colostrum, albeit in smaller quantities.

Due to the characteristics features thereof, exosomes can be considered “transporters”, therefore it was thought to exploit this ability thereof to enter other cells to transfer useful “loads” including chemotherapeutic and immunomodulatory agents, with the ability to direct the release thereof towards a given target. For example, exosomes allow crossing the blood-brain barrier and bringing the molecules of interest to the desired CNS sites. However, inserting these useful “loads” inside the vesicles requires the manipulation of the vesicles themselves (exogenous loading in which the small molecules are incorporated from the outside into the isolated EVs) or of the cells from which they come (endogenous loading in which the cell receives the necessary means to incorporate the small molecules in the EVs during the formation process).

Moreover, exosomes do not induce toxicity when injected repeatedly into mice and are well tolerated, thus the therapeutic use thereof is very promising.

There are several main methods for isolating exosomes: (1) ultracentrifugation, (2) size exclusion (e.g., ultrafiltration or chromatography), (3) immunological separation (e.g., antibody-bead capture), and (4) polymer-based precipitation.

The advantages in using each method vary based on the different origin of the exosomes, the degree of purity to be obtained (protein-exosome ratio, with the aim of minimizing the non-exosomal proteins which can be present in biofluids), yield or quality (preservation of the integrity and function of the exosomes).

For the purposes of the present patent, ultracentrifugation (UC) was used, recognized as the gold standard for the extraction and separation of exosomes and currently the most widely used isolation technique. UC allows separating and collecting the various components present in the original solution based on their differences in size and density, thus it is a suitable method for separating components with significant differences in the sedimentation coefficient from large sample volumes.

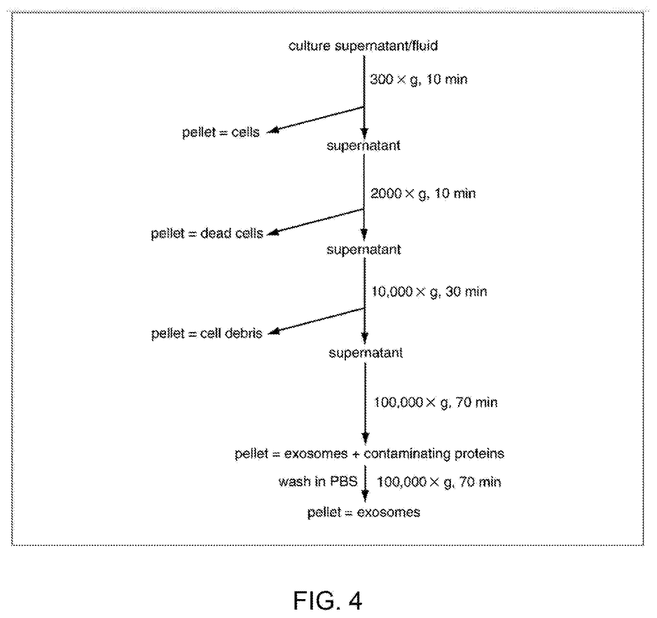

The UC method for isolating exosomes is mainly divided into two steps: first, a series of continuous centrifugations at medium-low speed to remove dead cells, cellular debris and large extracellular vesicles. The UC is then carried out at 100,000×g to separate the exosomes, followed by a series of washes in phosphate buffer (PBS), at the same UC speed, to remove impurities such as contaminating proteins. This method is summarized in the flow diagram in FIG. 4.

The inventors of the present patent application have surprisingly found that a highly concentrated exosome preparation is capable of enhancing the activity of the mixture of biological molecules of the present invention.

Moreover, even more surprisingly, the inventors of the present patent application have found that a highly concentrated exosome preparation from colostrum, rather than from other sources, is capable of further enhancing the activity of the mixture of biological molecules of the present invention.

In a preferred aspect of the present invention, the exosomes are isolated and concentrated from bovine colostrum.

The inventors of the present patent application have thus surprisingly found that in order for the activity of the mixture of bioactive molecules to be enhanced in all fields of application, a preferable condition is that the exosomes are isolated and concentrated from colostrum, rather than from other sources, and that the latter are added to the mixture in a functional amount.

For the purposes of the present invention, a highly concentrated exosome preparation is a preparation containing an amount of exosomes ≥1×10⁹/mL, preferably about 20-50×10⁹/mL.

In the preparations of the invention, the two components can be present, independently of each other, in the following amounts (in distilled water or saline):

| | |
|---|---|
| Exosomes | 5-200 × 10⁹/mL |
| Mixture of bioactive molecules from colostrum | 10-600 mg/mL |

In a preferred aspect of the invention, the two components can be present, independently of each other, in the following amounts:

| | |
|---|---|
| Exosomes | 10-100 × 10⁹/mL |
| Mixture of bioactive molecules from colostrum | 25-400 mg/mL |

For example, the two components can be present, independently of each other, in the following amounts:

| | |
|---|---|
| Exosomes | 20-50 × 10⁹/mL |
| Mixture of bioactive molecules from colostrum | 50-200 mg/mL |

In accordance with a second object, the present patent application describes formulations comprising a composition according to the invention.

In particular, such formulations are represented by: liquid solutions, lotions, foams, sprays, creams, salves, ointments, pastes, gels, membranes, clots, powders or other forms suitable for the medical use.

Such formulations can one or more additives, excipients and the other components necessary to obtain such formulations according to what is known in the art.

According to a preferred aspect of the present invention, the formulations described can optionally comprise one or more further effective components.

For example, one or more of the following can be included:

### Hyaluronic Acid

This can be present in various forms (esters, salts, hydrolysate, cross-linked, liposomal, etc.), among which sodium hyaluronate, the sodium salt thereof, in amounts between 5 and 50 mg/mL of distilled water or saline, preferably 25 mg/mL, is preferred.

Hyaluronic acid can further confer the ability to restore tissue tone and elasticity, as well as enhance and accelerate the re-epithelialization process.

### Amino Acids

In particular, glycine, L-proline, L-leucine and L-lysine are considered.

These can be present in a mixture, in amounts between 10 and 50 mg/mL of distilled water or saline, preferably 20 mg/mL.

Such amino acids can further confer the ability to stimulate collagen regeneration.

### Hydroxyapatite

Hydroxyapatite (HA) is the main inorganic component of bone and makes up 60-70% of the calcified skeleton and 98% of tooth enamel. It is biocompatible, binds quickly to adjacent hard and soft tissues and has a strong osteoconductive capacity.

The clinical indications of hydroxyapatite concern the reconstruction of bone tissue and the lining of endo-osseous dental implants to promote osseointegration. Hydroxyapatite is also used as a component of skin fillers.

In the case of bone reconstruction or dental implantology, the amounts used vary in relation to the surfaces to be reconstructed or covered.

In the case of fillers, 1 mL of gel is used containing from 20 to 50% of hydroxyapatite microspheres in 5 mL of distilled water or saline, preferably 40% of microspheres.

### β-Tricalcium Phosphate

β-tricalcium phosphate is a polycrystalline bioceramic, with osteoconductive properties, which in contact with water releases hydroxyapatite crystals. It is used for the resolution of deep intraosseous periodontal defects (periodontal regeneration), for filling post-extraction bone cavities, for bone regeneration, etc. β-tricalcium phosphate has a progressive resorption, unlike what occurs with hydroxyapatite, with a release of calcium and phosphorus ions which, for example in the case of intraosseous periodontal defects, contribute to the neo-apposition of bone, cement, and periodontal ligament. It is used in the amount of 1 g/mL of distilled water or saline.

### Platelet Concentrates

According to a further aspect of the present invention, the compositions of the invention can further comprise Platelet Concentrates (PCs).

In a preferred aspect of the invention, the platelet concentrates are autologous.

Autologous Platelet Concentrates (APCs) are blood components obtained by centrifuging the patient's blood in order to collect the most active components: platelets, fibrin and, in some cases, even leukocytes. The final product has a platelet concentration above the baseline level, therefore it has a higher number of bioactive molecules derived from the platelets themselves. The logic of the clinical use of such preparations is based on the concept of exploiting the thus enriched content thereof to stimulate different biological functions, such as chemotaxis, angiogenesis, proliferation and differentiation, so as to promote the healing of hard and soft tissues.

The use of the term Platelet-Rich Plasma (PRP) really began with Marx in 1998 when he published a comparative clinical study in which the regenerative potential of PRP was demonstrated in a series of patients undergoing mandibular reconstruction. PRP has then been associated with the concept of platelet growth factors and the potential contribution thereof in inducing tissue healing.

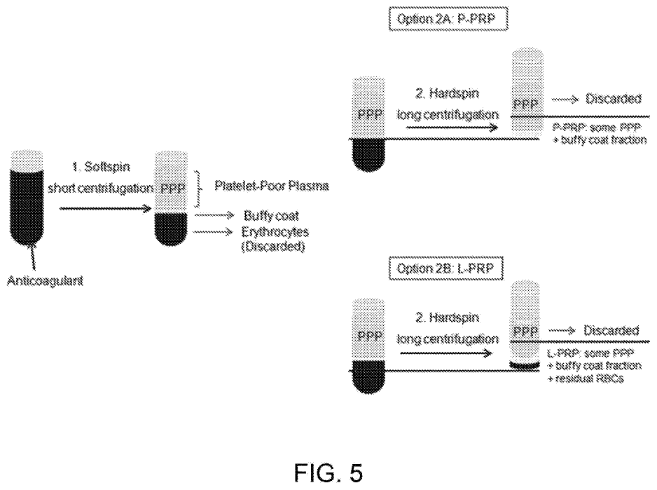

According to the protocol for the production of PRP, the patient's blood is collected in test tubes containing anticoagulants and processed by means of two centrifugation steps. FIG. 5 diagrammatically shows the specific protocol. The PRP thus obtained can be applied to the site to be treated with a syringe or activated by thrombin and/or calcium chloride to trigger platelet activation and stimulate fibrin polymerization.

After the collection of blood in test tubes with anticoagulant, the first low-intensity centrifugation (soft spin) allows the separation of the blood into three distinct layers: red blood cells at the bottom, a cell plasma (Platelet-Poor Plasma or PPP) at the top and a whitish layer referred to as the buffy coat, located therebetween, containing the highest concentration of platelets and leukocytes. For the production of Pure-PRP (P-PRP), the PPP and the buffy coat surface layer are transferred to another test tube and centrifuged at high intensity (hard spin), then most of the PPP and leukocytes are discarded and the P-PRP can be collected.

To obtain the leukocyte rich PRP (L-PRP), the PPP, the entire buffy coat layer and some residual red blood cells are collected and transferred to another test tube to be centrifuged at high intensity (hard spin), then most of the PPP is discarded thus obtaining an L-PRP containing the buffy coat with most of the platelets and leukocytes, some residual red blood cells and PPP.

There are currently more than 20 different commercial systems for preparing PRP which can lead to products with different features, in particular regarding the composition and the cellular concentration rate with respect to the baseline. On average, a 5-8× concentration is obtained, although a ratio of up to 11× has been reported with PRP.

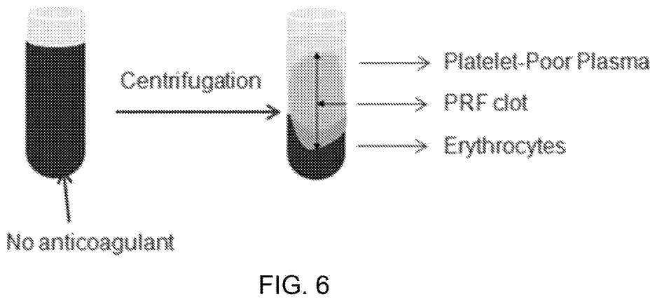

In 2001, a protocol was developed for producing a blood component referred to as Platelet-Rich Fibrin (PRF). In this case blood is collected in test tubes without anticoagulant and centrifuged at a moderate speed. Three layers are thus formed inside the test tube: red blood cells and acellular plasma are located, respectively, in the lower and upper part thereof, while the fibrin clot, positioned therebetween, forms the PRF (FIG. 6). Since the PRF clot naturally forms inside the test tube, it has a robust fibrin matrix in which most of the platelets and leukocytes are trapped.

Injectable PRF (i-PRF) has also been recently developed, which can be obtained with a further, even more delicate, centrifugation step. It has a liquid form, is very rich in white blood cells and can be used for infiltration into tissues and joints.

In parallel with the introduction of PRP and PRF, another platelet concentrate protocol referred to as Plasma Rich in Growth Factors (PRGF) was suggested in 1999.

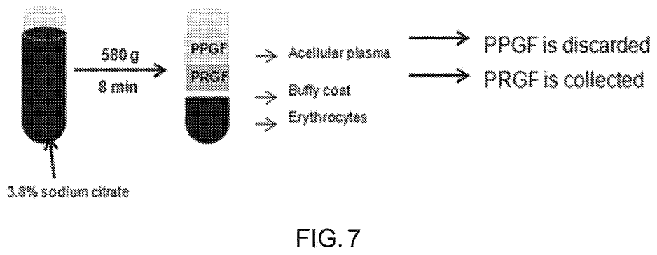

In short, blood is collected in test tubes with anticoagulant. After a low-intensity centrifugation, the red blood cells and the buffy coat layer are deposited on the bottom of the test tube and the plasma component on top of these. The latter is then manually separated into two fractions. The lower portion of about 2 mL, above the buffy coat, is PRGF, while the upper portion is Plasma Poor in Growth Factors (PPGFs) (FIG. 7). PRGF can be applied as a liquid fraction in the target site or it can be pre-activated by adding 0.2 mL of 10% CaCl₂) to induce clot formation.

Although the form thereof can vary based on the different applications (liquid form, gel, membranes or fibrin clots, etc.), as well as preparation protocols, there is multiple clinical evidence on the effectiveness of platelet concentrates in different fields of medicine.

In accordance with a third object of the present invention, the compositions and formulations detailed above are described for the medical use.

In particular, these can be advantageously used for the treatment of those conditions requiring tissue repair and regeneration.

For the purposes of the present invention, the application is in humans and animals.

In a preferred aspect, the compositions and formulations described find use in oral and maxillofacial regenerative surgical procedures; in orthopedics and sports medicine; in dermatology and aesthetic medicine; in gynecology; in andrology; in plastic and reconstructive surgery; in ophthalmology; in otolaryngology; in neuroscience; in the repair of organ injuries (brain, heart, lungs, liver, kidneys, larynx, pharynx, esophagus, etc.).

The same uses described above can be applied to the veterinary field.

The invention is described here in greater detail in the following experimental part.

### Example 1

### Topical Formulation (A)

| | Amount/mL |
|---|---|
| Ingredient | of distilled water or saline |
| Exosomes | 20 × 10⁹/mL |
| Mixture of bioactive molecules from | 50 mg/mL |
| colostrum | |
| Hyaluronic acid | 10 mg/mL |

The composition can possibly be modified by adding the following components (independently of each other):

| | Amount/mL |
|---|---|
| Ingredient | of distilled water or saline |
| Platelet-rich plasma (PRP) | 1 × 10⁶/μL (understood as the |
| | number of platelets/μL) |
| Hydroxyapatite | q.s. |
| β-tricalcium phosphate | 1 g/mL |

### Example 2

### Injection Formulation (B)

| | Amount/mL |
|---|---|
| Ingredient | of distilled water or saline |
| Exosomes | 20 × 10⁹/mL |
| Mixture of bioactive molecules from | 100 mg/mL |
| colostrum without immunoglobulins | |
| Hyaluronic acid | 25 mg/mL |

The composition can possibly be modified by adding the following components (independently of each other):

| | Amount/mL | |
|---|---|---|
| Ingredient | of distilled water or saline | |
| Platelet-rich plasma (PRP) | 1 × 10⁶/μL (understood as the | |
| | number of platelets/μL) | |
| Amino acids (mixture) | 20 | mg/mL |
| Hydroxyapatite (40% microsphere gel) | 200 | μL/mL |

### Example 3

### Proliferative and Regenerative Effectiveness Tests

In compliance with the principle of the 3Rs (Replacement, Reduction, Refinement) according to which the researcher should initially try, with the greatest possible effort, to replace or substitute the animal model thereof with an alternative model, all the proliferative and regenerative effectiveness tests of the compositions of the invention have been performed in vitro on cell cultures or on a biological model of human hair follicle, considering that the techniques replacing animal experimentation in regenerative medicine are highly effective and predictive.

In a first step all the compositions, based on a mixture of bioactive molecules and a functional amount of exosomes, were evaluated on a limited but highly significant number of cell cultures. The proliferative and regenerative responses obtained using the mixture of bioactive molecules from colostrum plus highly concentrated exosomes obtained from various sources (body fluids, tissues and cells, including adult mesenchymal stem cells) were then compared.

From the tests carried out, a superior proliferative and regenerative activity of the compositions comprising the exosomes isolated and concentrated from colostrum, with respect to the other sources, emerges. The entire test panel was then performed in vitro with the composition based on a mixture of bioactive molecules from colostrum+highly concentrated exosomes from colostrum.

The in vitro tests performed, to evaluate the effectiveness of the composition of the present invention in regenerative medicine, were as follows:
1. MTT Assay for Cell Viability and Proliferation.
2. Wound Healing Assay.
3. Test on a biological model of human hair follicle.

### 1. MTT Assay for Cell Viability and Proliferations

The MTT assay is used to measure the cellular metabolic activity, as an indicator of cell viability and proliferation. This colorimetric test is based on the reduction of a yellow tetrazolium salt (3-(4,5-dimethylthiazol-2-yl)-2,5-diphenyltetrazolium bromide or MTT) to purple formazan crystals by metabolically active cells. Vital cells contain NAD(P) H-dependent oxidoreductase enzymes which reduce MTT to formazan. The insoluble formazan crystals are dissolved using a solubilization solution and the resulting colored solution is quantified by measuring absorbance at 500-600 nanometers using a multi-well spectrophotometer. The darker the solution, the greater the number of viable and metabolically active cells.

This colorimetric test can be used for multiple applications, such as quantifying cell growth and viability and measuring cell proliferation in response to growth factors, cytokines, and nutrients.

For the execution of the MTT test, the following cell lines were used, necessary to demonstrate the proliferative and regenerative activity of the preparations of the present invention on the different tissues and organs.

Primary Epidermal Keratinocytes; Normal, Human, Adult (HEKa): keratinocytes are the most common type of skin cell, as they form the structural component of the epidermis.

Keratinocytes are used for scientific applications including studying the behavior of growth factors, wound repair, skin cancers, response to UV radiation, psoriasis, eczema, viral infections, cell differentiation, and cosmetics research.

Primary keratinocyte cultures have been extensively tested and validated to form skin equivalents which mimic the architectural features and behavior of normal skin cells.

Primary Dermal Fibroblast; Normal, Human, Adult (HDFa): fibroblasts play an important role in maintaining the structural integrity of connective tissue and protein synthesis of the extracellular matrix such as collagens, glycosaminoglycans and glycoproteins, molecules also involved in wound healing.

Fibroblasts are mediators in inflammation, cancer, angiogenesis, and pathological tissue fibrosis and are frequently used in studies related to wound healing, tissue engineering, and regenerative applications.

Therefore, the cell line of primary skin fibroblasts finds application in several fields of research: in the response to pathogens, skin ageing, wound healing and skin diseases, including scleroderma.

Primary Aortic Endothelial Cells; Normal, Human (HAEC): Endothelial cells form the tunica intima, the thin layer of cells lining the inner surface of blood vessels. This surface acts as a selective filter to regulate the passage of gases, fluids, immune cells and various molecules. Primary endothelial cell cultures can be isolated from the umbilical vein, aorta, pulmonary and coronary arteries and dermal vascular tissue and represent useful tools for the study of angiogenesis, cancer therapy, wound healing, burn therapy, tissue engineering and regeneration.

Primary Corneal Epithelial Cells; Normal, Human (HCEpC): primary corneal epithelial cell cultures are used as a model for studying eye re-epithelialization following photorefractive keratectomy (PRK) or Laser-ASsisted In situ Keratomileusis (LASIK). They are also used to study the regeneration of damaged corneas.

Primary Vaginal Epithelial Cells; Normal, Human (HVEC): primary vaginal epithelial cell cultures can be used to study the cellular physiology of the reproductive tract, the cellular response to infectious agents, and in other areas of research, including the regenerative activity of the damaged or atrophic vaginal epithelium.

Primary Skeletal Muscle Cells; Normal, Human (HSKMC): primary skeletal muscle cells are isolated from skeletal muscle and, cultured in vitro, provide an ideal model for studying the biology and metabolism of striated muscle cells, diabetes, insulin receptors, muscle tissue repair, and myotube development.

Primary Articular Chondrocytes; Normal, Human (NHAC-kn): primary articular chondrocytes are isolated from tissue taken from the knee. Joint cartilage is characterized by high wear resistance and poor regenerative qualities.

Primary articular chondrocyte cultures provide an ideal model for studying osteoarthritis, cartilage failure, inhibition of protein synthesis, and chondrocyte apoptosis.

Primary Osteoblasts; Normal, Human (NHOst): primary osteoblast cultures provide a homogeneous and rapidly proliferating model for the in vitro study of normal osteoblast differentiation, the physiology thereof, and the effects of hormones, growth factors, and other cytokines on osteoblast function and differentiation in vivo.

Primary Schwann Cells; Normal, Human (HSwC): in the peripheral nervous system, Schwann cells represent the main type of glial cells. The proliferation in vitro thereof can be stimulated by various growth factors including PDGF, FGF, neuregulin and others. Primary Schwann cell cultures provide a relatively simple, well-defined, and accessible model for studying neuropathies, nerve regeneration, and several diseases of nervous system development.

Primary Epidermal Melanocytes; Normal, Human, Adult (HEMa): primary melanocytes are specialized epithelial cells found primarily in the epidermis, where they produce the pigment melanin which gives skin the color thereof while protecting it from ultraviolet (UV) damage. Melanocytes are thus useful in research into pigmentation disorders and for the development of effective therapies for such disorders, in particular malignant melanoma.

Primary epidermal melanocyte cultures are used for the in vitro study of wound healing and as a model for toxicity and irritability studies, for studies of skin response to UV radiation, psoriasis and other skin diseases and also in cosmetic research (e.g., skin lightening compounds and skin protective compounds).

Primary Hepatocytes; Normal, Human (HH): primary hepatocytes are often referred to as the “gold standard” for in vitro liver tests. Most of the specific cellular functions of hepatocytes can be maintained in culture, which makes them the best model for replicating liver function in vitro, e.g., metabolism, liver toxicity, cell biology, and host-pathogen interactions. Primary hepatocyte cultures are thus a cost-effective and economical alternative to in vivo testing.

Primary Small Airway Epithelial Cells; Normal, Human (HSAEC): primary small airway epithelial cell cultures provide an ideal model for studying respiratory infections, asthma, chronic obstructive pulmonary disease, cystic fibrosis, inflammation response, harmful effects of smoking.

Primary Renal Epithelial Cells; Normal, Human (HREC): primary renal epithelial cell cultures provide an ideal model for research related to hypertension, diabetes, oncology, renal fibrosis, inflammation, autoimmune diseases, drug screening, and toxicology.

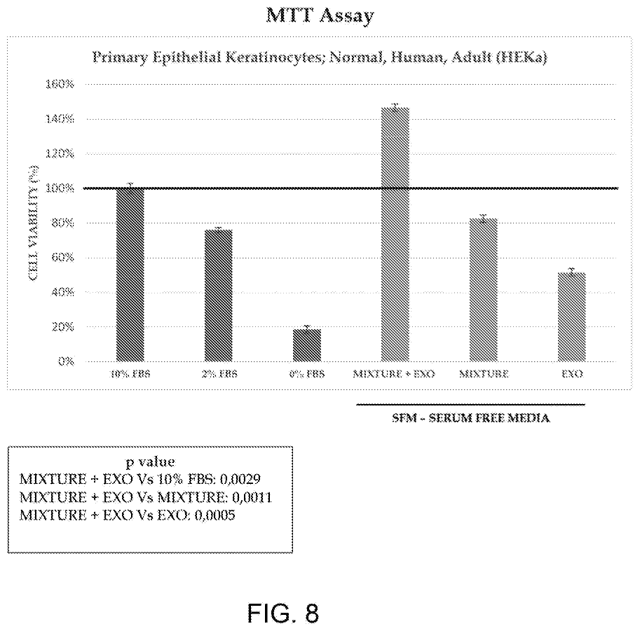

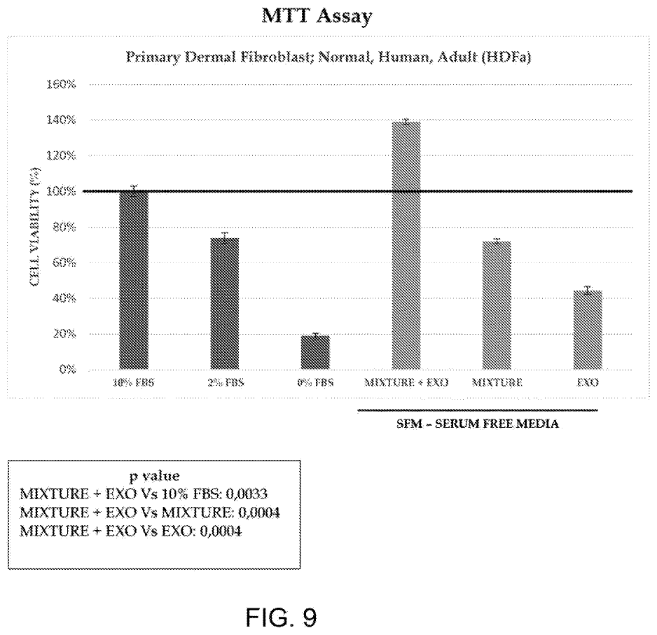

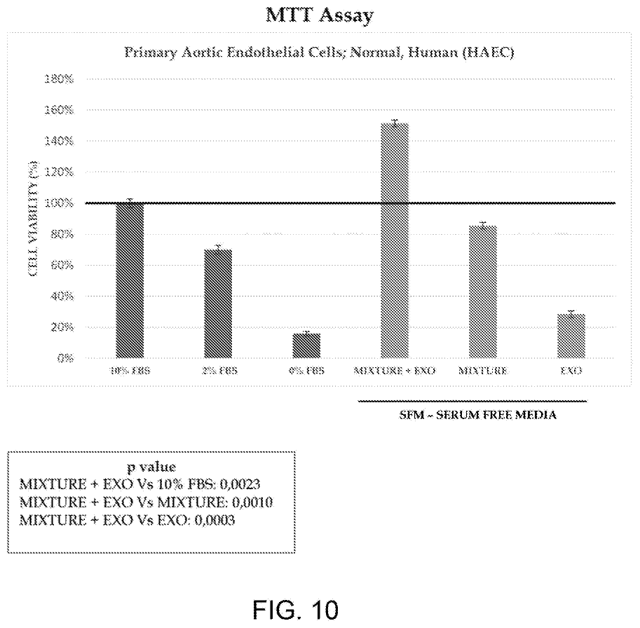

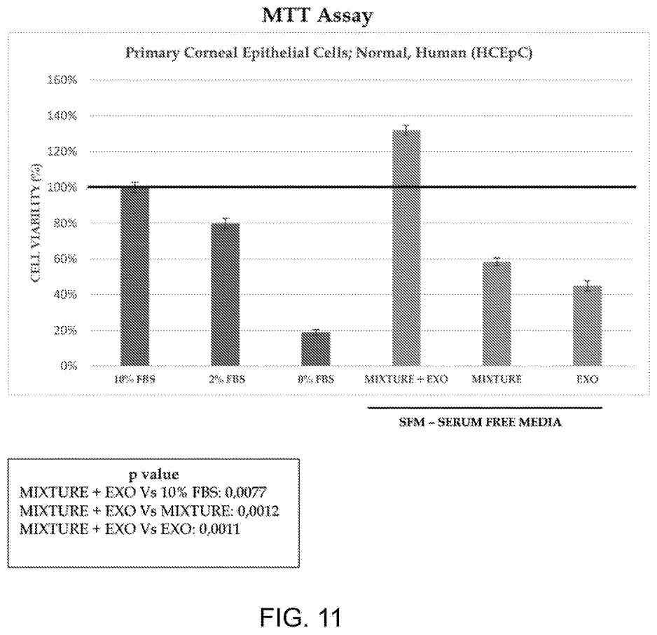

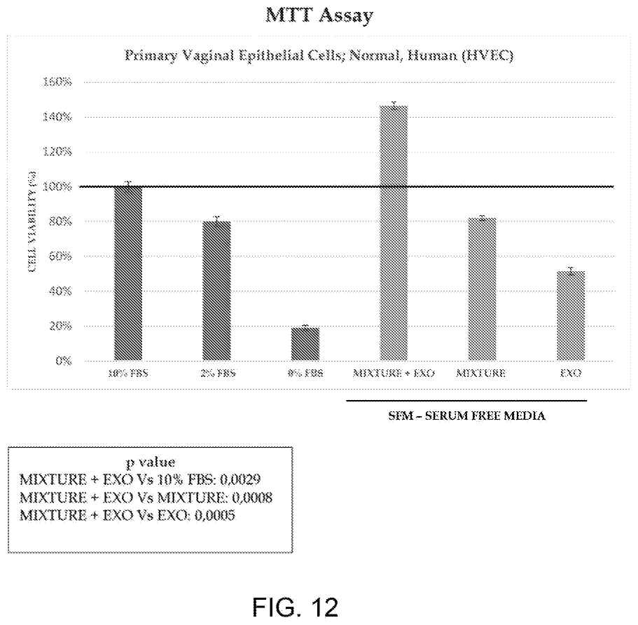

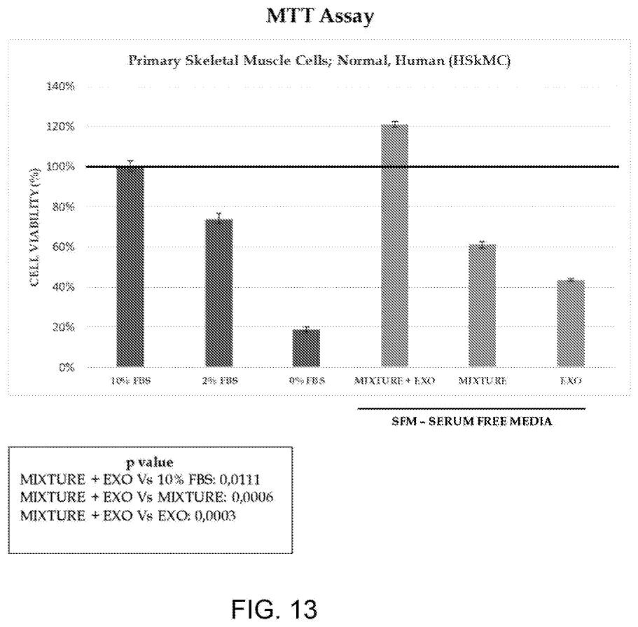

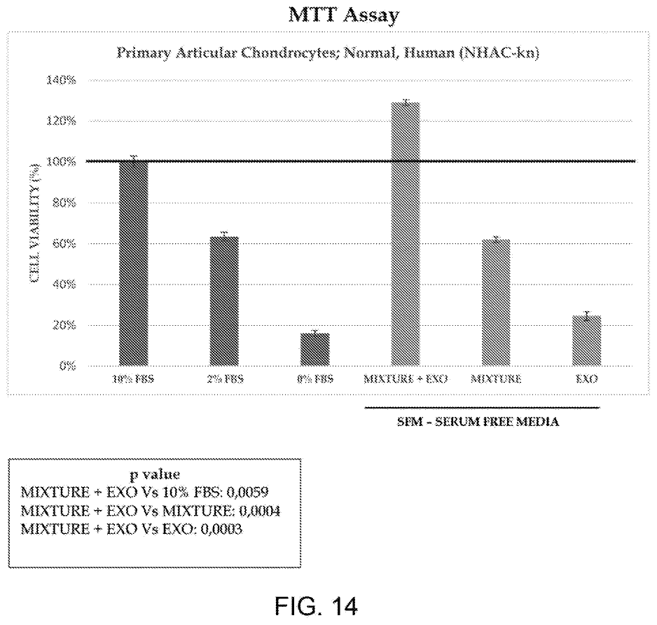

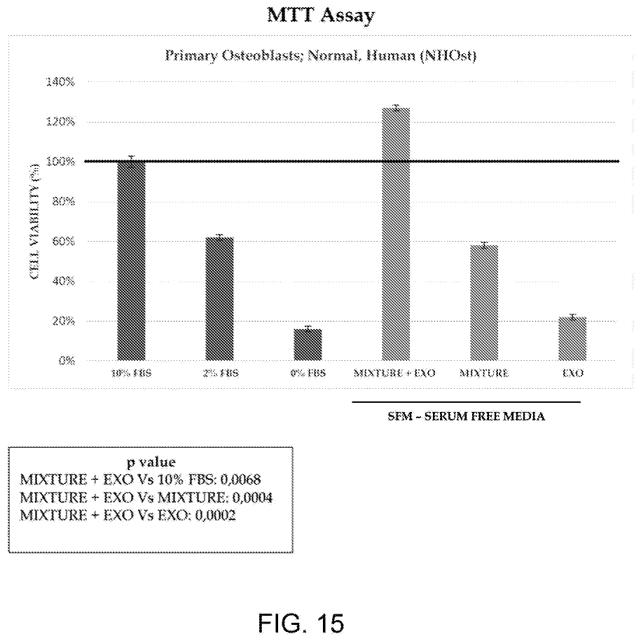

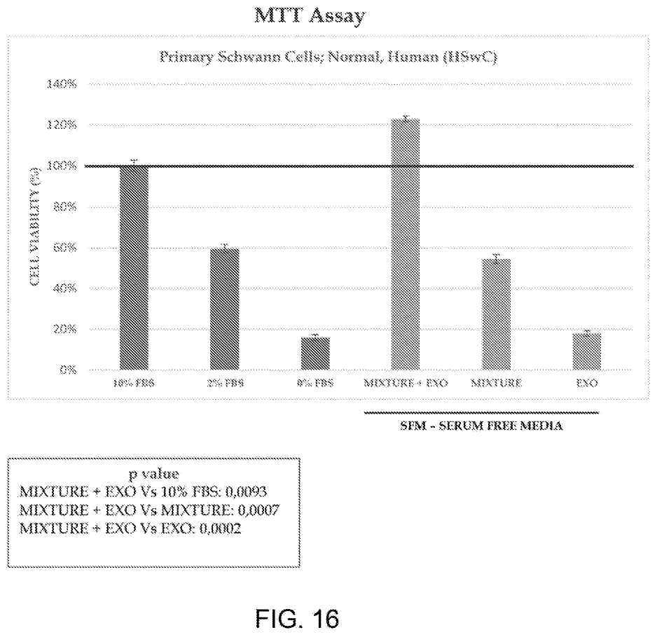

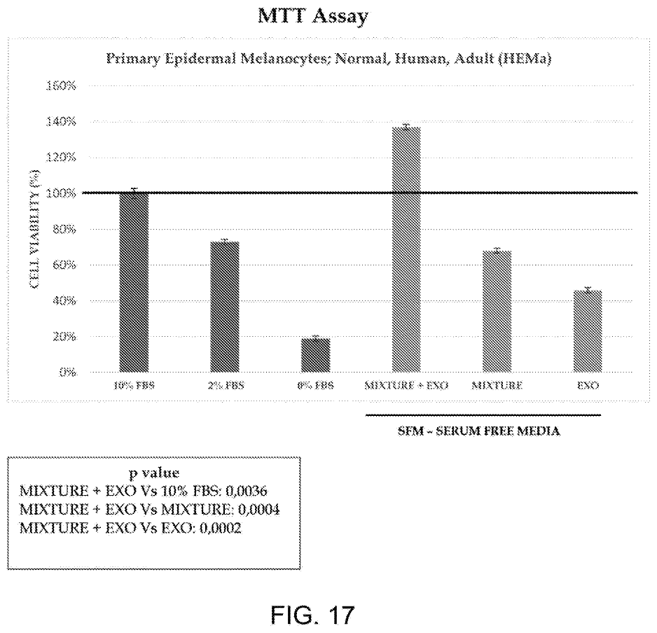

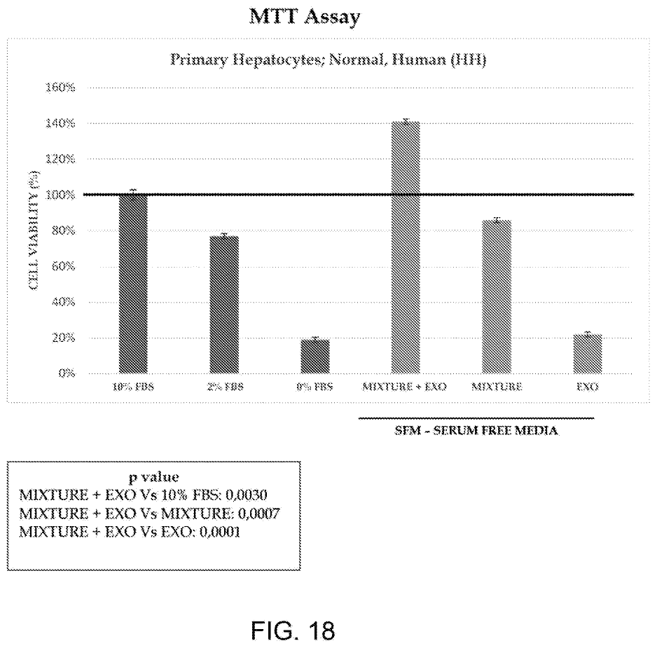

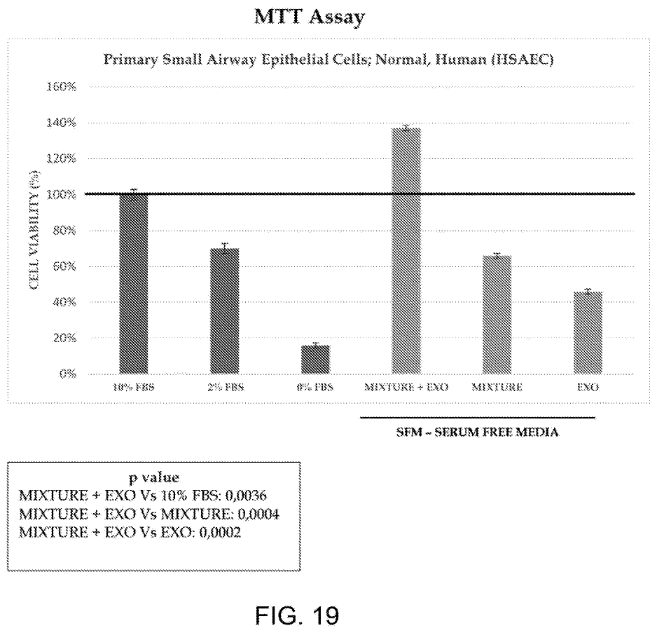

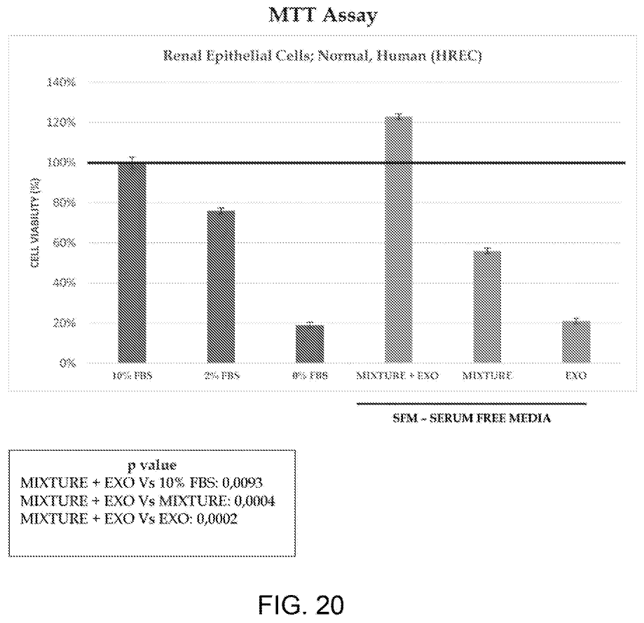

The results obtained with the MTT Assay for Cell Viability and Proliferations, using the cell cultures described above, are shown in FIG. 8 to FIG. 20.

### 2. Wound Healing Assay

The wound healing assay, also known as the “scratch test,”, is a well-established two-dimensional (2D) technique which can be used to study wound healing in vitro. This method was one of the first to be developed for the study of cell migration and measures the speed with which cells, in a cell monolayer, migrate to fill a free space.

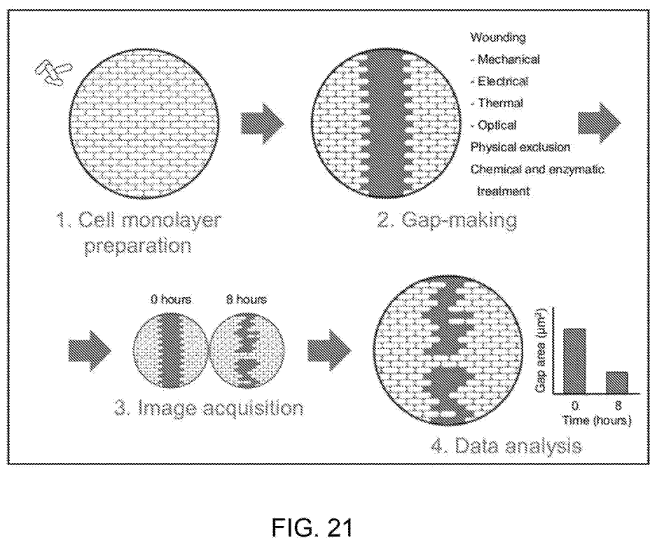

The technique reproduces the wound by creating an empty space in a confluent cellular monolayer and consists of four main steps which are described below (FIG. 21 shows an overview thereof).

### 1. Cell Culture Preparation

The first step of the test is the culture of a confluent cell monolayer. This monolayer represents the in vivo condition of the tissue prior to injury, as an intact epithelium. Epithelial or endothelial cells are normally used to produce the monolayer. Human corneal epithelial cells (HCEpC) were used to evaluate the effectiveness of the composition of the present invention in regenerative medicine.

### 2. Scratch-Making

After the cells have become confluent, the next step is to create a cell-free space in the monolayer, usually by means of mechanical scratching.

### 3. Data Acquisition

Optical microscopy using phase contrast imaging can be used to observe cells migrating into the wound area.

Once the microscope is set up, a series of time-lapse images (snapshot method) can be acquired as the cells migrate into the cell free space. These time points are then collected at 24, 48 and 72 hours after the start of the experiment. The migration images can then be used to collect measurements or be evaluated visually.

### 4. Data Analysis—“Scratch” Closing

Once the gap closure images have been acquired, different analysis methods can be used to quantify the cell migration rate. The method used for the purposes of the present patent calculates the change in wound area over time as a percentage of wound closure.

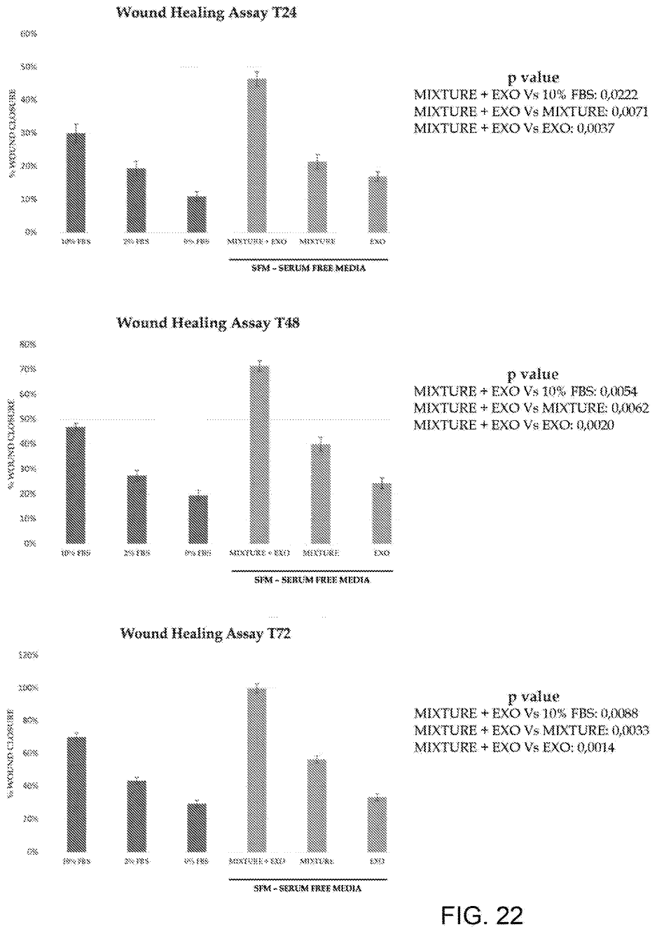

The results of the Wound Healing Assay, used to evaluate the effectiveness of the composition of the present invention in wound healing, are shown in FIG. 22.

### 3. Test on a Biological Model of Human Hair Follicle

Hair grows in cycles. Starting from the growth phase (anagen), the hair follicle and the shaft thereof go through the regression phase (catagen), the resting phase (telogen) and finally the exogenous phase (shedding phase). The hair follicles used in this test show a growth phase (anagen) of 15 days before moving on to the regression phase (catagen).

The biological model used for this test is represented by hair follicles micro-sectioned from fragments of human skin. The purpose of the test is to verify the growth of the hair follicle in the presence or not of the substances to be tested: the hair follicles were thus cultured in the William culture medium without the addition of other compositions (negative control), in the same culture medium with the addition of the composition of the present patent, i.e., the composition based on a mixture of bioactive molecules from colostrum+highly concentrated exosomes from colostrum (positive control), or with the addition of the other two reference products (mixture alone or exosomes alone). Hair follicle growth was followed for 18 days, with measurements of the length thereof being taken at regular intervals.

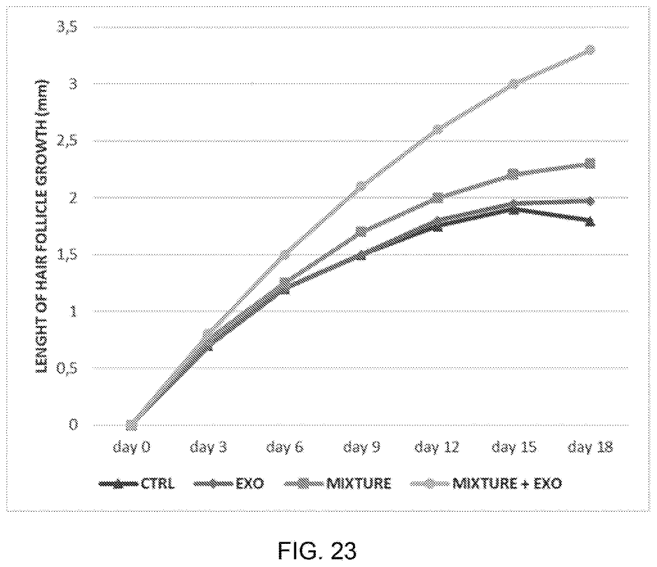

The results of this test, used to evaluate the effectiveness of the composition of the present invention in the treatment of alopecia, are shown in FIG. 23.

### Proliferative and Regenerative Effectiveness

The in vitro tests performed to evaluate the proliferative and regenerative effectiveness of the composition of the present invention in the multiple medical uses are summarized below.
a) To evaluate the effectiveness of the composition of the present invention in oral and maxillofacial regenerative surgical procedures, the in vitro tests were performed using the Wound Healing Assay and the MTT Assay with HEKa, HDFa, HAEC cells (healing of post-extractive alveoli, treating periodontal defects, endodontics and endodontic surgery) and with NHOst cells (dental implantology, elevation of the maxillary sinus).
b) To evaluate the effectiveness of the composition of the present invention in orthopedics and sports medicine, the in vitro tests were performed using the MTT Assay with NHAC-kn cells (treating osteoarthritis, treating degenerative disease of the intervertebral discs); with NHOst, HAEC cells (managing fractures and nonunions); with HDFa cells (treating tendon injuries, enhancing the post-surgery healing process of tendons and ligaments) and with HSKMC cells (treating muscle injuries).
c) To evaluate the effectiveness of the composition of the present invention in dermatology and aesthetic medicine, the in vitro tests were performed using the MTT Assay with HEKa, HDFa cells (skin rejuvenation, treating acne, treating stretch marks, treating melasma, treating periorbital hyperpigmentation); with HEMa cells (treating vitiligo), as well as with the “Test on a biological model of human hair follicle” for treating alopecia.
d) To evaluate the effectiveness of the composition of the present invention in gynecology, the in vitro tests were performed using the Wound Healing Assay and the MTT Assay with HVEC, HDFa cells (treating skin injuries, healing surgical wounds, treating vaginal atrophy and vaginal dryness, treating vulvar dystrophy, aesthetic gynecology in vaginal rejuvenation).
e) To evaluate the effectiveness of the composition of the present invention in andrology, the in vitro tests were performed using the MTT Assay with HAEC cells (treating erectile dysfunction).
f) To evaluate the effectiveness of the composition of the present invention in plastic and reconstructive surgery, the in vitro tests were performed using the Wound Healing Assay and the MTT Assay with HEKa, HDFa, HAEC cells (treating burns, treating wounds and skin ulcers, treating keloids and hypertrophic scars post-excision); and with HDFa, HSkMC cells (breast reconstruction).
g) To evaluate the effectiveness of the composition of the present invention in ophthalmology, the in vitro tests were performed using the Wound Healing Assay and the MTT Assay with HCEpC cells (treating persistent corneal epithelial defects, treating corneal abrasions and ulcers, corneal surgery, treating dry eye syndrome, treating ocular surface syndrome after Laser-ASsisted In situ Keratomileusis-LASIK or after photorefractive keratectomy-PRK).
h) To evaluate the effectiveness of the composition of the present invention in otolaryngology, the in vitro tests were performed using the Wound Healing Assay and the MTT Assay with HEKa, HDFa, HAEC cells (treating atrophic rhinitis, treating tympanic membrane perforation).
i) To evaluate the effectiveness of the composition of the present invention in neuroscience, the in vitro tests were performed using the MTT Assay with HSwC cells (treating traumatic brain injuries, treating traumatic spinal cord injuries, treating neurodegenerative and autoimmune diseases of the CNS, treating peripheral nerve injuries).
l) To evaluate the effectiveness of the composition of the present invention in the repair of organ injuries (brain, heart, lungs, liver, kidneys, larynx, pharynx, esophagus, etc.) resulting from ischemia, infectious diseases, degenerative diseases, fibrosis, radiotherapy treatments, traumatic injuries, burns, etc., the in vitro tests were performed using the MTT Assay with HH, HSAEC, HREC cells, in addition to what has already been reported for the individual tissues forming the different organs.

The in vitro tests described above to evaluate the proliferative and regenerative effectiveness of certain substances on human cells/tissues/organs are comparable to those used for animals, thus all tests performed to evaluate the effectiveness of the composition of the present invention in human medicine and surgery are also to be considered valid in veterinary medicine and surgery.

In all the in vitro tests performed, the results obtained with the composition of the present patent application (i.e., the composition based on the mixture of bioactive molecules+highly concentrated exosomes) were far superior to the results obtained with all the other products tested or with the reference sample (control), both in terms of proliferative and regenerative effectiveness (MTT Assay, FIGS. 8 to 20; Wound Healing Assay, FIG. 22; Test on a biological model of human hair follicle, FIG. 23).

Student's t (p<0.05 significant values; p<0.01 highly significant values) was used to compare the results obtained with the in vitro tests. The statistical calculations show that the difference in the post-treatment averages between the composition of the present patent, the other products tested and the reference sample (control) is significant and, in almost all cases, highly significant.

The above description further demonstrates the greater effectiveness of the composition based on a mixture of bioactive molecules plus highly concentrated exosomes, of the present invention, with respect to the other tested products (mixture of bioactive molecules alone, highly concentrated exosomes alone). Such a greater effectiveness is clearly higher than what could be expected from the mere arithmetic sum of the results obtained by the individual products.

In particular, it has been surprisingly found that by combining the exosomes with the bioactive molecules of the mixture, the correct growth and regeneration of tissues is modulated and essential nourishment is provided to cells in the growth and proliferation phase.

From the above description of the present invention, the advantages given by the suggested formulations will be immediately apparent.

In particular, it has been observed that the effectiveness of these formulations is closely linked to the synergy of the two components (mixture of bioactive molecules and highly concentrated exosomes) and the functional interdependence thereof; in fact, the bioactive molecules of the mixture act synergistically with the exosomes and the content thereof (membrane proteins, cytosolic and nuclear proteins, extracellular matrix proteins, lipids, mRNA, miRNA and DNA nucleic acids, growth factors, cytokines and enzymes) exponentially increasing the activity thereof.

Moreover, it appears that this interaction results in an increase in the half-life of the bioactive molecules of the mixture, which would otherwise be very short, and/or an increase in the diffusion rate thereof in the intercellular spaces. Both of these factors would contribute to resolving the defect of transport of the bioactive molecules of the mixture through the healthy tissues to those areas where the action thereof would be required.

By virtue of this synergy, the bioactive molecules of the mixture can act, promoting the reparative process; this occurs by stimulating neoangiogenesis, even in poorly vascularized tissues, and the regeneration of damaged tissues which are replaced with cells of the same type, thus avoiding the replacement thereof with connective tissue (fibrosis). The application of the composition of the present invention thus prevents the onset of hypertrophic or keloid scars, which represent one of the most common wound healing disorders.

It should be noted that for the exosomes of the present invention, the only clearly demonstrated function, i.e., the function of transporters, is excluded, since for the execution of the tests described in the experimental part no manipulation of the exosomes was carried out in order to incorporate the bioactive molecules therein.

It was also observed that the exosomes of the present invention remain in a living and vital state even up to 6-8 months and even in the absence of lyophilization, thus demonstrating a high and unexpected stability.

In order to meet contingent and specific needs, those skilled in the art may make adaptations and modifications to the invention described above, and may replace components with other similar ones, without however departing from the scope of the claims set forth below.

For example, one or more excipients can be modified according to the pharmaceutical form selected.

1. A composition comprising a mixture of bioactive molecules and exosomes, wherein said mixture is isolated from colostrum.

2. The composition of claim 1, wherein said mixture of bioactive molecules is in an amount of about 10-600 mg/mL in distilled water or saline.

3. The composition of claim 1, wherein the exosomes are in an amount of at least 1×10⁹/mL.

4. The composition of claim 1, wherein said exosomes are in an amount of about 5-200×10⁹/mL.

5. The composition of claim 1, wherein said mixture is isolated from human or animal colostrum.

6. The composition of claim 1, wherein said mixture is isolated from colostrum collected before a newborn has had the opportunity to nurse and empty the udder of the first fraction produced, regardless of time elapsed since the moment of birth.

7. The composition of claim 1, wherein said mixture comprises growth factors, cytokines, stem cell stimulating factors, complement proteins, antibacterial and antiviral factors, and immunoglobulins.

8. The composition of claim 1, wherein said mixture is at least 90% free of IgG, IgA, and IgM immunoglobulins.

9. The composition of claim 1, wherein the exosomes are isolated and concentrated from human or animal cells, tissues or body fluids.

10. The composition of claim 9, wherein said exosomes are isolated from colostrum.

11. The composition of claim 1, wherein the exosomes contain membrane proteins, cytosolic and nuclear proteins, extracellular matrix proteins, lipids, nucleic acids (mRNA, miRNA and DNA), growth factors, cytokines and enzymes.

12. The composition of claim 1, further comprising one or more compounds selected from the group consisting of: hyaluronic acid, platelet concentrates, amino acids, hydroxyapatite, and β-tricalcium phosphate.

13. The composition of claim 1, wherein said composition is a topical composition or an injection composition.

14. The topical composition of claim 13, having the following formulation:

| | Amount/mL |
|---|---|
| Ingredient | of distilled water or saline |
| Exosomes | 20 × 10⁹/mL |
| Mixture of bioactive molecules | 50 mg/mL |
| from colostrum | |
| Hyaluronic acid | 10 mg/mL |

and further comprising, or as an alternative to hyaluronic acid, one or more of the following ingredients:

| | Amount/mL |
|---|---|
| Ingredient | of distilled water or saline |
| Platelet-rich plasma (PRP) | 1 × 10⁶/μL (understood as the |
| | number of platelets/μL) |
| Hydroxyapatite | q.s. |
| β-tricalcium phosphate | 1 g/mL |

15. The injection composition of claim 13, having the following formulation:

| | Amount/mL |
|---|---|
| Ingredient | of distilled water or saline |
| Exosomes | 20 × 10⁹/mL |
| Mixture of bioactive molecules | |
| from colostrum without | 100 mg/mL |
| immunoglobulins | |
| Hyaluronic acid | 25 mg/mL |

and further comprising, or as an alternative to hyaluronic acid, one or more of the following ingredients:

| | Amount/mL | |
|---|---|---|
| Ingredient | of distilled water or saline | |
| Platelet-rich plasma (PRP) | 1 × 10⁶/μL (understood as the | |
| | number of platelets/μL) | |
| Amino acids (mixture) | 20 | mg/mL |
| Hydroxyapatite (40% | 200 | μL/mL |
| microsphere gel) | | |

16. A topical or injection formulation comprising the composition of claim 1, in a form selected from the group consisting of: liquid solutions, lotions, foams, sprays, creams, salves, ointments, pastes, gels, membranes, clots, and powders.

17. (canceled)

18. A method of treating a condition requiring tissue repair and regeneration in a subject in need thereof, the method comprising administering to the subject the composition of claim 1.

19. The method of claim 18, wherein the condition requiring tissue repair and regeneration is an oral or maxillofacial surgery related condition selected from the group consisting of: healing of post-extraction alveoli, treatment of periodontal defects, endodontics and endodontic surgery, dental implantology, and maxillary sinus lift.

20. The method of claim 18, wherein the condition requiring tissue repair and regeneration is an orthopedic condition selected from the group consisting of: osteoarthritis, degenerative disease of intervertebral discs, tendon injuries, post-surgery healing process of tendons and ligaments, muscle injuries, fractures and nonunions.

21. The method of claim 18 wherein the condition requiring tissue repair and regeneration is a dermatologic condition selected from the group consisting of: skin aging, acne, stretch marks, melasma, periorbital hyperpigmentation, vitiligo, and alopecia.

22. The method of claim 18, wherein the condition requiring tissue repair and regeneration is a gynecologic condition selected from the group consisting of: vaginal atrophy, vaginal dryness, vulvar dystrophy and vaginal rejuvenation.

23. The method of claim 18, wherein the condition requiring tissue repair and regeneration is erectile dysfunction.

24. The method of claim 18, wherein the condition requiring tissue repair and regeneration is a plastic or reconstructive surgery condition selected from the group consisting of: treatment of burns, treatment of wounds, treatment of skin ulcers, treatment of keloids and hypertrophic scars post-excision, and breast reconstruction.

25. The method of claim 18, wherein the condition requiring tissue repair and regeneration is an ophthalmic condition selected from the group consisting of: persistent corneal epithelial defects, corneal abrasions and ulcers, dry eye syndrome, ocular surface syndrome after Laser-Assisted In situ Keratomileusis (LASIK) or after Photorefractive Keratectomy (PRK).

26. The method of claim 18, wherein the condition requiring tissue repair and regeneration is an otolaryngology condition selected from the group consisting of: atrophic rhinitis, and tympanic membrane perforation.

27. The method of claim 18, wherein the condition requiring tissue repair and regeneration is a neurologic condition selected from the group consisting of: traumatic brain injuries, traumatic spinal cord injuries, neurodegenerative and autoimmune diseases of the CNS, and peripheral nerve injuries.

28. The method of claim 18, wherein the condition requiring tissue repair and regeneration is organ injury resulting from: ischemia, infectious diseases, degenerative diseases, fibrosis, radiotherapy treatments, traumatic injuries, and burns.

29. The composition of claim 1, wherein said mixture is at least 95% free of IgG, IgA, and IgM immunoglobulins.

30. The composition of claim 1, wherein said mixture is at least 99% free of IgG, IgA, and IgM immunoglobulins.

31. The composition of claim 1, wherein said mixture is totally free of IgG, IgA, and IgM immunoglobulins.
